(19) United States
(12) Patent Application Publication
(10) Pub. No.: US 2019/0022388 A1
(43) Pub. Date: Jan. 24, 2019

(54) DEVICE AND SYSTEM TO MEASURE AND ASSESS SUPERFICIAL MUSCLE CONTRACTILE CHARACTERISTICS

(71) Applicant: Quanimus Inc., Toronto (CA)

(72) Inventor: Brent STUCKE, Oakville (CA)

(21) Appl. No.: 16/069,077

(22) PCT Filed: Feb. 3, 2017
(86) PCT No.: PCT/CA2017/000022

§ 371 Date: Jul. 10, 2018

Related U.S. Application Data
(60) Provisional application No. 62/291,996, filed on Feb. 5, 2016.

(51) Int. Cl. A61N 1/36; A61B 5/11; A61B 5/00; A61N 1/04

(52) U.S. Cl. CPC A61N 1/36031; A61B 5/1107; A61B 5/6833; A61N 1/0452; A61N 1/025; A61N 1/0492; A61N 1/36003; A61B 2562/046; A61B 2562/0219; A61N 1/0476

(57) ABSTRACT

The present invention relates to a device and system to measure and assess superficial skeletal muscle mechanical and neuromuscular contractile characteristics, and interpret the results to provide metrics with quantifiable and qualitative descriptors relating to muscle function. The present device provides a further type of mechanomyography and a new use for acceleromyography by measuring the mechanical muscle movement of an involuntary stimulated muscle from an automated electro-stimulation protocol to determine muscle contractile properties. Muscle twitch response during the latent, contraction and relaxation phase is measured using an array of multiple accelerometers on a sensor pad to assess and diagnose muscle. function from various measurements. This information is processed using algorithms to determine muscle function abnormalities, muscle activation patterns, muscle symmetry of lateral muscle pairs, muscle synchronization of antagonist muscle, muscle force, muscle acceleration, muscle speed, muscle tone, muscle fatigue, muscle power/torque and muscle efficiency.

### FIELD OF THE INVENTION

The present invention relates to the field of biomechanics and more specifically to the measurement and assessment of superficial skeletal muscle mechanical and neuromuscular contractile characteristics.

### BACKGROUND OF THE INVENTION

Some muscle tests image aspects of muscle structure (ultrasonography, magnetic resonance imaging, compute tomography/other imaging studies), while others measure aspects of muscle function (electromyography, mechanomyography, force plate analysis, other force transducer technologies). These technologies, devices and measurement techniques measure aspects of muscle structure, nerve conduction, motor unit recruitment, and force production in an attempt to measure how muscle contractions individually, and/or as systems, act to produce joint motion and therefore function and performance. They are used to establish states of normalcy whereby states of abnormal or dysfunctional criteria can then be identified. They are additionally used to try to quantify muscular function/performance as it relates to different individuals from different populations and for different standards of use: For most healthcare professionals and individuals who want this kind of information, gaining access to valuable, objective information about muscle function has not been readily or conveniently available to them in a low cost manner. Historically, muscle testing has been limited to diagnostics that are time consuming to administer, have long waiting periods in certain regions due to high demand, are expensive to purchase and expensive to operate/administer, require highly trained technicians to interpret, and are at times invasive and painful to the subject.

There are a number of technologies that measure skeletal muscle movements in a non-invasive manner. Some of these technologies include the technology used in the TOF-Watch® that measures acceleration, the technology in the Myoton® that measures muscle oscillation, the technology in TMG S1 (tensiomyography) that measures muscle displacement, the technology used in the Myotonometer® that measures muscle stiffness, and the technology used in the Neutone™ that measures muscle hardness. Some of these technologies provide muscle information and characteristics either through active or passive assessment; however, as a result of dependency on device placement by the technician, results are often difficult to accurately repeat. Additionally, factors that can influence the state of the muscle prior to or during assessment are rarely explored and documented (formally known as context-based information). This makes interpretation of the test results difficult and also impacts the reproducibility of the test results.

There are several devices which measure the functionality of a muscle. These devices use many different technologies as a way of quantifying an aspect of a muscle contraction. The devices also measure muscles in various conditions of activation (voluntary/involuntary), thresholds of activation (submaximal/maximal/supramaximal), contraction conditions (static/dynamic), contraction types (isometric/isokinetic), and a number of muscle contractile properties (mechanicophysiological, electrophysiological, metabolic). These devices also use a number of descriptors (quantifiable/qualiflablk, parameters/metrics) coupled with relative and absolute comparisons to provide a range of values that represents a continuum of muscle measurements that covers the population of abnormal to normal to elite muscle function. This range of values can be selected by the technician administering the test to represent different populations based on sport participation or other shared population characteristics to give more accurate comparisons and interpretation of the test results.

For example, tensiomyography uses a large tripod and tripod arm, to house a single force sensor with a spring loaded probe which is placed on a muscle. Specifically, the probe is placed on a small precise area of the muscle belly which measures the maximal radial displacement to measure the response of the muscle in an electro-stimulated state. However, this technology only collects muscle response data in one dimension rather than collecting all three dimensions of muscle movement data. The value of tensiomyography is also heavily dependent on the technician's expertise to repeatedly place the force sensor at the appropriate angle in the appropriate spot on the muscle belly to acquire meaningful information, which is often a time consuming exercise and makes it difficult to have repeatable and accurate measurements due to human error, motion and mechanical artifact. As a result, many tests fail and have to be repeated, consuming more time. It is time consuming for the technician to move the tripod, tripod arm and single sensor to test each muscle, as well as requiring significant training for a technician.

There is a substantial gap between expensive medical devices/technologies that statically assess an individual's muscle structure (i.e. MRI and Ultrasound) and subjective in-clinic dynamic assessments that evaluate an individual's muscle function (i.e. movement screens, manual muscle tests, and orthopedic tests). Healthcare practitioners, medical researchers, sports science and human performance professionals lack quantitative, objective, muscle function and neuro-muscular function data on an individual's muscle that is reliable, affordable and easy to obtain through muscle diagnostic testing. There is a need for a device that objectively, selectively, quantifiably, accurately, quickly and cost effectively measures superficial skeletal muscle mechanical and neuromuscular contractile characteristics to determine repeatable changes in muscle function.

### SUMMARY OF THE INVENTION

In an embodiment of the present invention, a superficial skeletal muscle response measuring system is provided comprising two sensor pads each with a plurality of three-axis accelerometers capable of measuring muscle contraction responses to stimulation, a pair of electro-stimulation electrodes for each sensor pad, a control box having capability to send stimulation to said electro-stimulation electrodes and to process and transmit data when muscle contraction responses are received from said sensor pads. The superficial muscle response measuring system may comprise three-axis accelerometers capable of measuring submaximal, maximal and supramaximal muscle contraction responses to stimulation.

In an embodiment of the present invention there is provided a sensor pad comprising a circuit board with a three by three array of nine accelerometers, a housing body in which the circuit board rests, and a protective covering over the circuit board. The circuit board may additionally comprise nine cells within which the accelerometers fit. The sensor pad may additionally comprise an adhesive layer under said housing body, said adhesive comprising the quality of adhering to and releasing from skin.

In a further embodiment of the present invention there is provided a sensor pad comprising a circuit board with a three by three array of nine accelerometers, a housing body in which the circuit board rests, and a protective covering over the circuit board, wherein the circuit board is carved out to further comprise slots around each accelerometer wherein each accelerometer rests on a cantilever arm. The protective covering may additionally comprise nine cells within which said accelerometers fit. The sensor pad may additionally comprise an adhesive layer under said housing body, said adhesive comprising the quality of adhering to and releasing from skin. The sensor pad may additionally comprise corresponding contiguous slots in the covering, the housing body, and the adhesive layer.

In a further embodiment of the present invention there is provided a sensor pad comprising a circuit board with a four by four array of sixteen accelerometers, a housing body in which the circuit board rests, the housing body having sixteen cells in which said accelerometers fit within when the circuit board is resting in the housing body, and a protective covering over the circuit board.

In an embodiment of the present invention there is a control box comprising a first connector for connecting to a first pair of electrodes, a second connector for connecting to a second pair of electrodes, a third connector for connecting to a first sensor pad with multiple accelerometers capable of measuring superficial muscle contraction responses, and a fourth connector for connecting to a second sensor pad with multiple accelerometers capable of measuring superficial muscle contraction responses, wherein the control box is capable of controlling the delivery of stimulation to said two pairs of electrodes and receiving muscle contraction responses from the two sensor pads.

The sensor pads for the control box are capable of measuring at intervals within contraction and relaxation phases of a muscle contraction response with 3-axis accelerometers.

The superficial muscle response measuring system of the present invention, in which said control box further comprises software with a first algorithm to identify from said nine accelerometers of each of said sensor pads a selected group ranging from one to nine accelerometers from each of said sensor pads with the most reliable force, acceleration, velocity and distance data, and a second algorithm, combining data from the x, y, z axis of each of said selected group of from each of said sensor pads to produce three combined results of each of force, acceleration, velocity and distance.

The superficial muscle response measuring system, further comprising computer software hosted on the cloud with an algorithm for characterizing and averaging the data from at least two of three combined results into a single result of force, acceleration, velocity and distance for each of said sensor pads.

In use, the superficial muscle response measuring system determines muscle function normalities and abnormalities, muscle activation patterns, lateral symmetry of muscle pairs, muscle co-contraction of agonist-antagonist muscles, muscle force, muscle acceleration, muscle velocity, muscle distance, muscle tone, muscle fatigue, and muscle efficiency.

### BRIEF DESCRIPTION OF THE FIGURES

These and other aspects of the present invention will be apparent from the brief description of the drawings and the following detailed description in which:

FIG. 1 is an exploded view of a sensor pad assembly of a first embodiment of the present invention.

FIG. 2 is a cross-sectional view of the sensor pad assembly of FIG. 1.

FIG. 3 is an exploded view of a sensor pad assembly of a second embodiment of the present invention.

FIG. 4 is a cross-sectional view of the sensor pad assembly of FIG. 3.

FIG. 5 is an exploded view of a sensor pad assembly of a third embodiment of the present invention.

FIG. 6 is a cross-sectional view of the sensor pad assembly of FIG. 5.

FIG. 7 is a depiction of a sensor pad assembly of a third embodiment of the present invention in use on legs of a person along with the accompanying components of electro-stimulation electrodes, control box, local computing devices and virtual cloud.

FIG. 8 is a perspective view of a control box of an embodiment of the present invention.

FIG. 9 is a block diagram of hardware of the control box of FIG. 8.

FIG. 10 is a block diagram view of electronic components of the sensor pad assembly of FIG. 5.

FIG. 11 is a perspective view of a sensor pad and a pair of electro-stimulation electrodes connected to a control box in an embodiment of the present invention of FIG. 5.

FIG. 12 is a flow chart of a ramping protocol of an embodiment of the present invention.

### DETAILED DESCRIPTION OF THE INVENTION

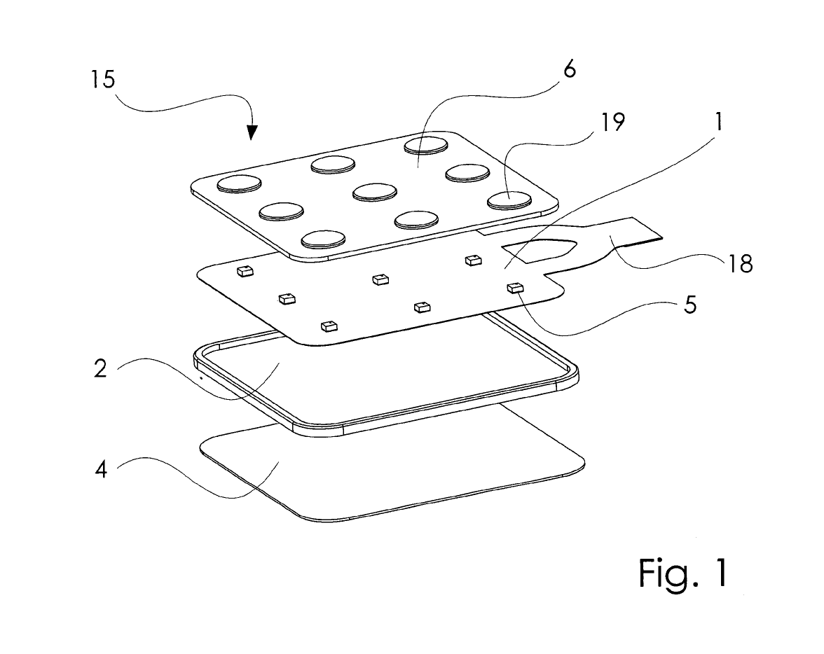

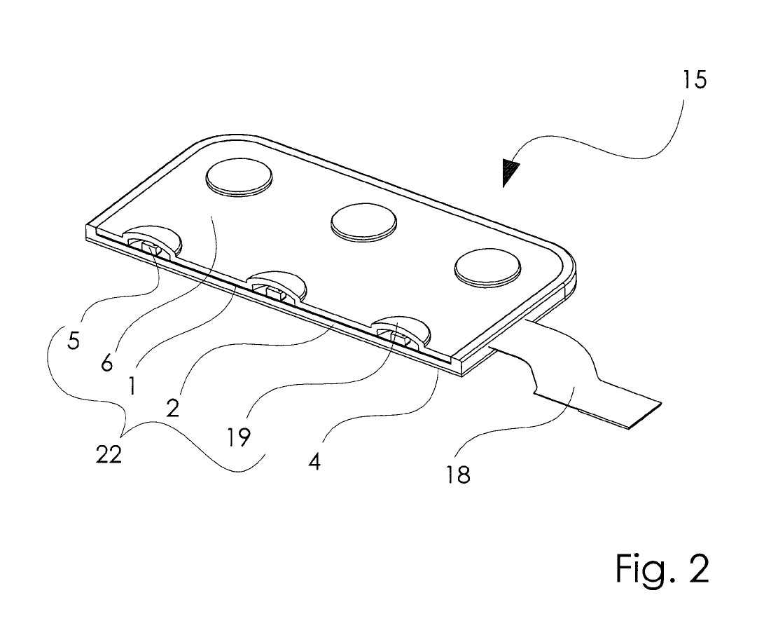

FIGS. 1 and 2 show a sensor pad assembly 15 of a first embodiment of the present invention. The sensor pad assembly 15 comprises nine, three-axis accelerometers 5 connected to a flexible circuit board 1 comprising a tail 18 at one end. The accelerometers 5 and flexible circuit board 1 comprise a sensor array.

A sensor array assembly 22 comprises: the covering 6; the flexible circuit board 1; the accelerometers 5; and a flexible housing body 2.

The flexible housing body 2 is shaped to house the flexible circuit board 1. The flexible housing body 2 protects the sensor array assembly 22 from being damaged. A flexible protective covering 6 comprises nine cells 19 such that the accelerometers can rest within the cells 19

A disposable hydrogel adhesive layer 4 is applied to the underside of the housing body 2 to keep the sensor array assembly 22 in place when used on a person. The adhesive provides a bond with a person's skin. The hydrogel adhesive layer 4 can be disposed of and replaced when no longer sufficiently adhering to skin.

In use, the sensor pad assembly 15 is first oriented over a muscle body and then placed with the hydrogel adhesive layer against the skin in order to maintain correct orientation on a person's skin.

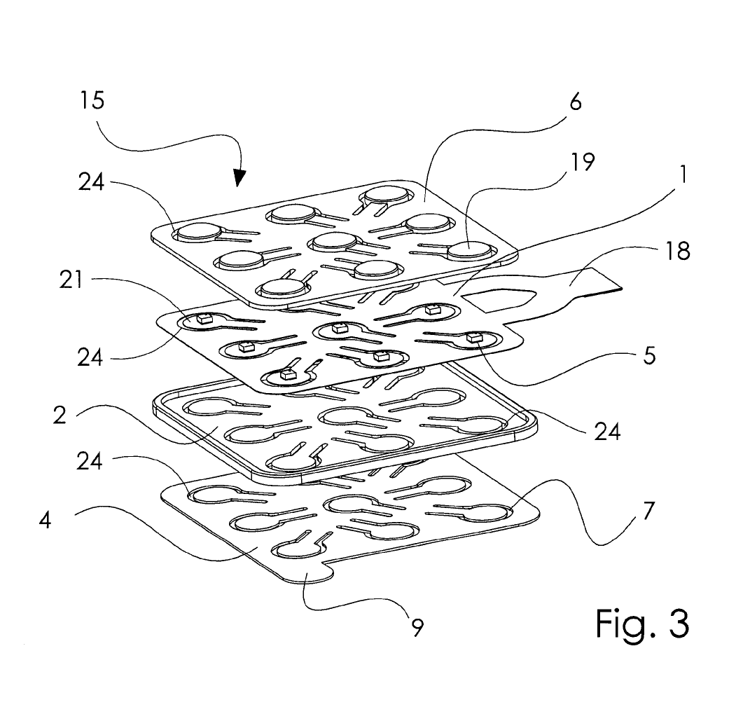

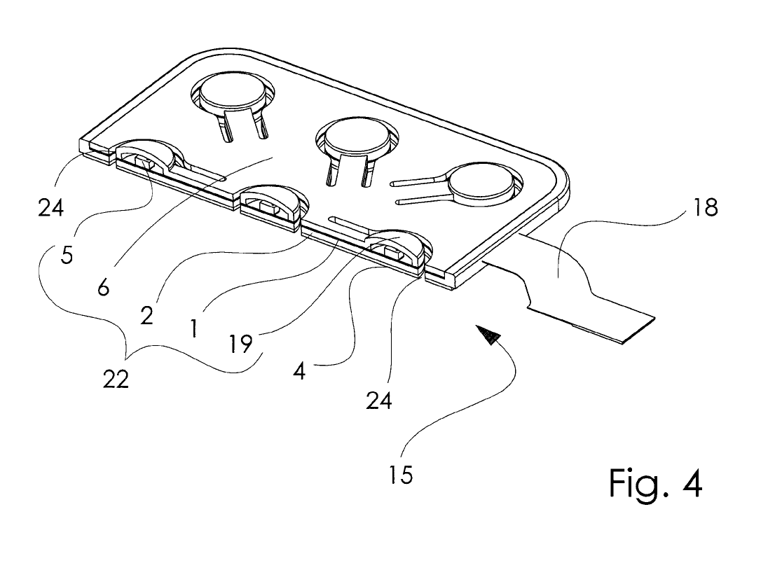

FIGS. 3 and 4 show a sensor pad assembly 15 of a second embodiment of the present invention. The sensor pad assembly 15 comprises nine, three-axis accelerometers 5 connected to a flexible circuit board 1 comprising a tail 18 at one end. The accelerometers 5 and flexible circuit board 1 comprise a sensor array.

A flexible housing body 2 is shaped to house the flexible circuit board 1. A flexible protective covering 6 comprises nine cells 19 such that the accelerometers can rest within the cells 19.

A sensor array assembly 22 comprises: the covering 6; the flexible circuit board 1; the accelerometers 5; and the flexible housing body 2.

A disposable hydrogel adhesive layer 4 is applied to the underside of the housing body 2 to keep the sensor array assembly 22 in place when used on a person. The adhesive provides a bond with a person's skin. The hydrogel adhesive layer 4 can be disposed of and replaced when no longer sufficiently adhering to skin.

In use, the sensor pad assembly 15 is first oriented over a muscle body and then placed with the hydrogel adhesive layer against the skin in order to maintain correct orientation on a person's skin.

In this second embodiment the accelerometers 5 are on individual cantilever arms 21 carved in the flexible circuit board 1. The carved out portion around each cantilever arm 21 form slots 24, which slots 24 may be also found in the covering 6, the housing body 2 and the hydrogel adhesive layer 4 such that the slots 24 are contiguous through the layers. In the second embodiment of the present invention each accelerometer 5 is isolated on its respective cantilever arm 21 and each cantilever arm 21 conforms to the contour of the muscle below it.

A pull tab 9 on the hydrogel adhesive layer 4 is included in this second embodiment and could be included in the first embodiment as well. When the sensor array assembly is adhered to the skin, this pull tab 9 can be detached from the skin, and the sensor array assembly can be removed by pulling the pull tab 9.

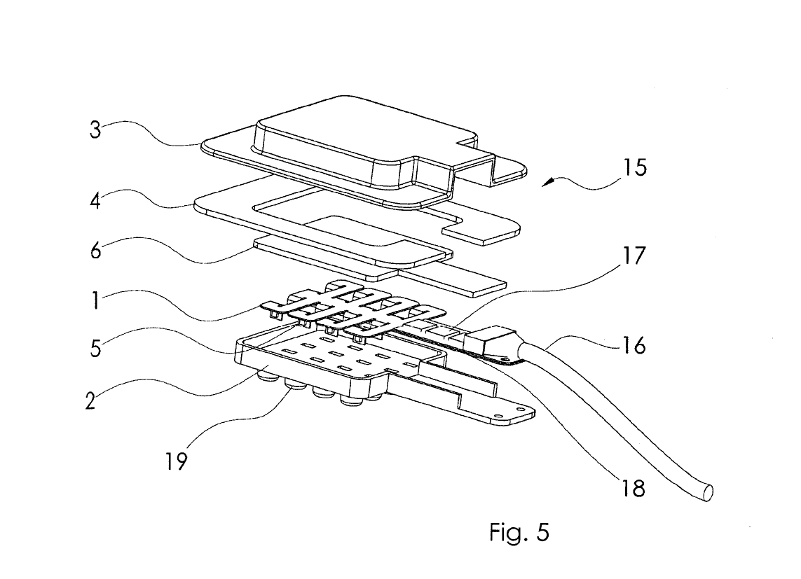

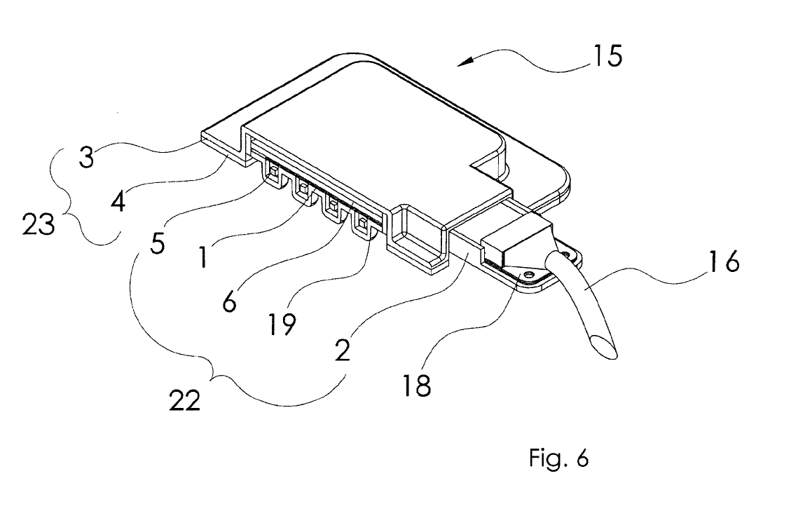

FIGS. 5 and 6 show a sensor pad assembly 15 of a third embodiment of the present invention with means for connecting to a control box (not shown). The sensor pad assembly 15 comprises sixteen, three-axis accelerometers 5 connected to a flexible circuit board 1. The accelerometers 5 and flexible circuit board 1 comprise a sensor array. The flexible circuit board 1 is joined to a connector 16 for an eight conductor cable. There are three chips 17 located on the tail 18 of the flexible circuit board 1. These chips 17 receive data from the accelerometers 5 and convert it to a signal that is relayed to the control box (not shown).

A flexible housing body 2 shaped to house the flexible circuit board 1 comprises sixteen cells 19 such that the accelerometers can rest within the cells 19. A flexible protective covering 6 formed by pouring a layer of electronic potting material provides a cover over the circuit board 1 to protect the sensor array from being damaged.

A sensor array assembly 22 comprises: the covering 6; the flexible circuit board 1 with all of its components including the accelerometers 5; and the flexible housing body 2.

A disposable mounting cap 3 with a hydrogel adhesive layer 4 applied to the underside together form a sticky cap 23 to maintain the sensor array assembly 22 in place when used on a person. The sticky cap 23 covers the sensor array assembly 22 and holds it in position since the adhesive provides a bond with a person's skin. As such, a characteristic of the adhesive is that it can be adhered to the skin as well as releasing from the skin. The mounting cap 3 with hydrogel adhesive layer 4 can be disposed of and replaced when no longer sufficiently adhering to skin.

In use, the sensor array assembly 22 is first oriented over a muscle body and then the sticky cap 23 is placed over the sensor array assembly 22 in order to maintain correct orientation on a person's skin. The sticky cap 23 is friction fit over the sensor array assembly 22 and the resulting sensor pad assembly 15 remains in position on a person's skin.

As will be understood by those skilled in the art, the accelerometers are not required to be enclosed in cells (such as the cells 19 in the covering 6 of the first and second embodiments of the sensor pad assembly, and the cells 19 in the housing body 2 of the third embodiment of the sensor pad assembly), but covering the accelerometers in a manner that protects them from damage keeps them in working order.

In an embodiment of the present invention, the three-axis accelerometers 5 measure acceleration of a person's muscle in 3 coordinates (X,Y,Z). The accelerometers 5 used in an embodiment of the invention may have 8G maximum in each axis.

It was determined that there is an about 25 mm area in a muscle belly, which will be referred to as the “representative sweet spot”, which represents what is happening in the muscle belly and which an experienced technician can find, or find after trial and error. In order to cover that representative sweet spot, in an embodiment of the present invention the sensor pad is 50 mm by 50 mm to provide sufficient coverage over the muscle belly being measured. Further the density of the spacing between multiple accelerometers is seventeen mm center to center. In an embodiment of the invention there are nine accelerometers spaced seventeen mm center to center in a 50 mm by 50 mm sensor pad.

In a further embodiment of the invention there are sixteen accelerometers spaced 12 mm center to center in a 50 mm by 50 mm sensor pad.

Although the embodiments shown in the figures all depict a “universal” sensor pad having a predetermined size, number of accelerometers, and centre to centre spacing of accelerometers in that sensor pad, it is understood that the sensor pads of the present invention may be of different sizes and have varying numbers of accelerometers since in a further embodiment of the invention the feedback from the sensors is filtered so that only the maximum signal received from one or more accelerometers is utilised. In this aspect of the invention the sensor pad configuration may vary since only the maximum signal readings will be used, for example, in a sensor pad with nine accelerometers, only one accelerometer may give a good result and be used (however, it's also possible that all nine accelerometers could give a maximum signal and all readings will then be used). It is further understood the centre to centre distance and number of accelerometers may be greater or smaller than shown in the figures depending on the pad configuration. For example, in a three by three array of nine accelerometers, the accelerometers may be spaced 20 mm apart from centre to centre and in a 5 or 6 accelerometer configuration may be spaced 10 mm to 20 mm apart from centre to centre. It is also understood that the distance between the accelerometers can also vary.

Other possible configurations could include, but are not limited to: 5 accelerometers arranged in a two by two configuration with a central accelerometer with a pad measurement of 55 mm by 55 mm; 6 accelerometers arranged in a three by two configuration with a pad measurement of 50 mm by 33 mm; or 4 accelerometers arranged in a two by two configuration with a pad measurement of 25 mm by 25 mm. Moreover, it is understood that the pad could take the form of any shape and is not limited to a square or rectangular pad as shown in the figures and described herein. Bigger people and/or bigger muscles may require a bigger sensor pad and smaller people and/or smaller musclea may require a smaller sensor pad. For example, a sensor pad for the tibialis anterior may use a sensor pad assembly with 3 accelerometers in a sensor pad about 15 mm×15 mm.

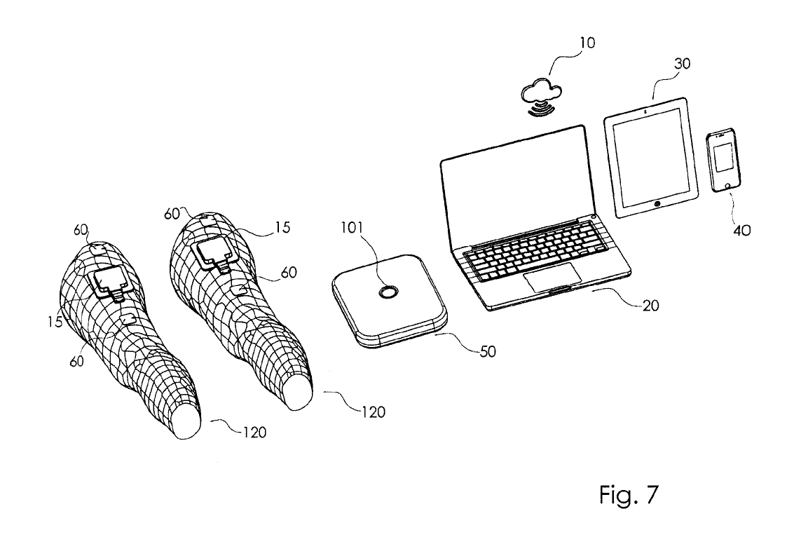

FIG. 7 shows an embodiment of a system of the present invention, in which one sensor pad assembly 15 of the third embodiment of the invention and two electro-stimulation electrodes 60 are placed on each leg 120. In this embodiment, the sensor pad assembly is part of a system, with electro-stimulation electrodes 60, a control box 50, a choice of local computing devices 20, 30, 40 and the virtual cloud 10. Test results are stored on a cloud application 10 or may be stored locally. A computing device, such as a laptop 20, tablet 30, or cellular device 40 (such as a smart device or cell phone) operates the e-stimulation and testing software.

The control box 50 interacts with a cellular device app or with computer software to deliver electrode stimulation to two muscle bellies simultaneously, while receiving accelerometer information from sixteen times two accelerometers, each producing values on all three axis. In an embodiment, the control box 50 has an emergency manual shut down switch 101 (accessible by both the person being treated and the operator). In this illustration, a person's legs 120 are shown but the sensor pad assembly 15 and electrodes 60 can be applied to any muscle of the body. The electro-stimulation electrodes 60 are in direct contact with a person's skin to provide electrical stimulus to contract the muscle.

In this embodiment of the invention, two sensor pad assemblies 15 are placed on each leg 120, and after stimulation the sensor pad assemblies 15 “sense”, and the app or software records muscle movement in three axis (x, y, z). In another embodiment of the invention, one sensor pad assembly 15 and two electro-stimulation electrodes 60 can be placed on one muscle belly only, however, this provides half the muscle movement results.

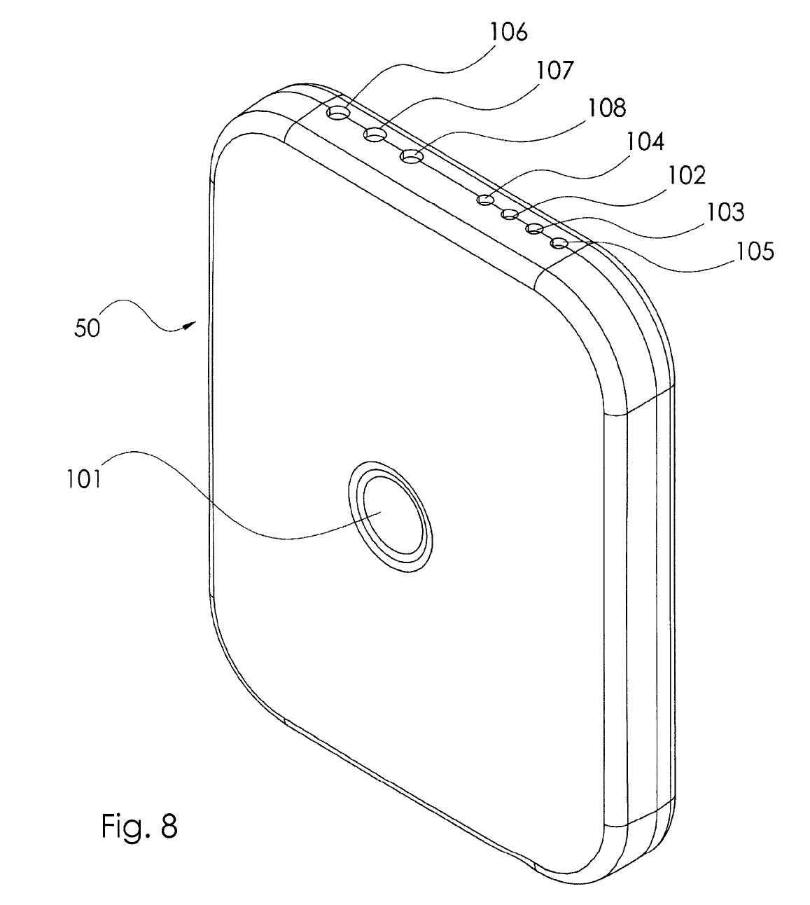

FIG. 8 shows a control box 50 of an embodiment of the present invention, wherein one control box 50 controls two sensor pad assembly systems, each system having two electrodes and one sensor pad assembly 15. There are various connectors on the control box 50. A first electro stimulation electrode connector 102 is a connector for a stimulation cable (not shown) which runs to a first pair of electro stimulation electrodes 60 (not shown), and a second electro stimulation electrode connector 103 is a connector for a cable (not shown) which runs to a second pair of electro stimulation electrodes 60 (not shown). A first sensor pad assembly connector 104 is a connector for a cable (not shown) which runs to a first sensor pad assembly 15 (not shown) and a second sensor pad assembly connector 105 is a connector for a cable (not shown) which runs to a second sensor pad assembly 15 (not shown). The ‘power in’ connector 106 is a connector for an external power cable (not shown) in order to provide power to the control box 50, but there is also an internal back up battery (not shown in FIG. 8). A manual shut down switch 101 on the control box stops a test in progress when pressed, which provides a physical disconnect of the power to the electrostimulation electrodes 60 shown in FIG. 7. An external antenna connector 107 provides a mount for an external Bluetooth® antenna (or other wireless receiver device). The main power switch 108 turns the control box on or off.

It is understood that it is possible that one control box can also control a sole sensor pad assembly. It is also understood that one control box could control more than two sensor pad assemblies simultaneously, for example, by adding more channels to control the additional sensor pads.

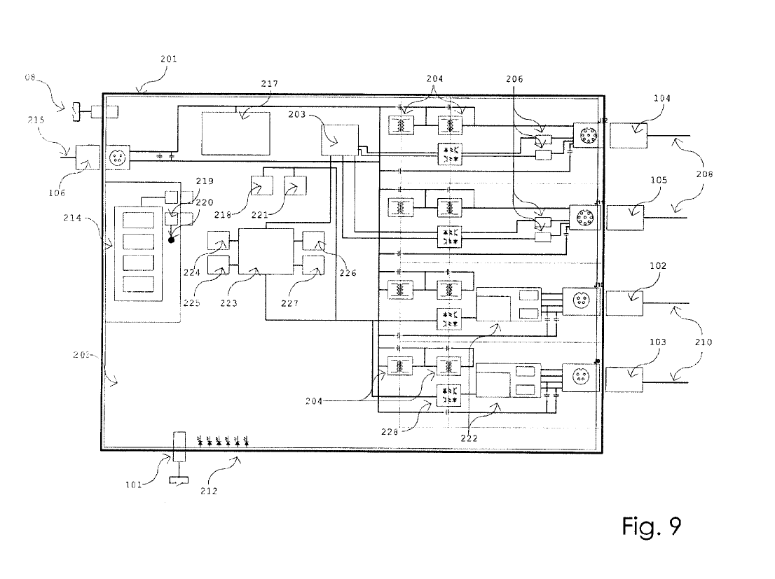

FIG. 9 is a block diagram view of the control box hardware of the control box 50 used with the third embodiment of the sensor pad assembly 15. The inside of the control box 50 has an outer plastic enclosure 201 containing the control hardware and battery, and an inner rigid PCB (printed circuit board) 202 supports all of the electronics within the control box and provides electrical connections between parts. A quad high-speed UART (universal asynchronous receiver/transmitter) 203 quadruples the data-transmission capabilities from the sensor pad assemblies.

A first isolation barrier and second isolation barrier are made up of isolation components 204 which provide up to 5000 VAC isolation between system electronics and circuits connected to a person receiving treatment via the electrodes and sensor pad assemblies.

An LVDS IC (low-voltage differential signalling, integrated circuit) 206 provides differential signaling capability between the sensor pad assembly 15 and the control box 50 and allows for greater distances between the control box 50 and the sensor pad assembly 15. The first and second sensor pad assembly connectors 104 and 105 are touch-proof, locking, keyed connectors for cables 208 which each connect to a sensor pad assembly. Cables 208 are flexible cable to allow for maximal mobility of a sensor pad assembly in relation to the control box 50. The electro stimulation electrode connectors 102 and 103 are touch-proof, keyed connectors from a control box 50 to two electro stimulation electrodes (not shown) via stimulation cables 210.

The LEDs (light emitting diodes) 212 are indicator LEDs which can be included within a control box to indicate information about the state of the control box 50. In an embodiment of the invention the LEDs can provide the following examples of status indicators: no illumination indicates off-blue illumination indicates standby mode; green illumination indicates stimulation mode; and amber illumination indicates stop mode. As will be understood, variations of colour can be used and the control box of the present invention does not require LEDs to indicate information about the control box.

The control box 50 has a manual shut down switch 101, which is shown in FIGS. 7 and 8, to provide a physical means to initiate a stop at the device in case of software failure or other reason. The ‘power in’ connector 106 is a connector for an external power cable 215. Power is provided in an embodiment of the invention at 24V DC by means of a medical grade power cable 215, and the main power switch 108 turns the power on and off at the control box 50. Voltage regulators 217 regulate the 24V to working levels for the variety of circuits and integrated circuits in the control box. A lithium ion battery 214 provides long run time between charges, battery connector 219 connects to the battery 214, charger circuits 218 provides a means to charge the battery safely, battery temperature monitor 120 monitors the temperature of the battery and raises fault if the temperature is over a critical value, and gas gauge 221 monitors the power usage through the battery 214 to estimate its charge level.

Stimulation circuits 222 are used to generate an electrical stimulation to the muscles of a person being assessed using programmed pulse characteristics, and a JTAG (joint test action group) programming header 224 allows for programming of the control box 50. A microcontroller 223 provides control over the control box and the sensor pad assemblies. RAM (rapid active memory) module 225 buffers the collected sensor data before being sent out from the control box 50 to the local device 20, 30 or 40. A Bluetooth® module facilitates a connection between the control box 50 and the local device 20, 30 or 40 which transmits the data after connection has been established over Bluetooth®. WiFi module 227 is an alternative connectivity between the control box 50 and the local device 20, 30, or 40.

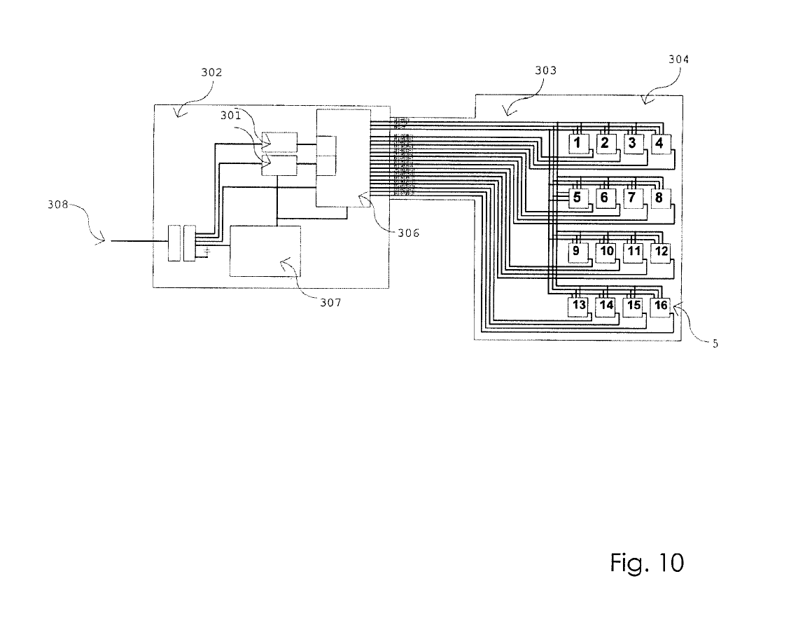

FIG. 10 is a block diagram of the electronic components of the third embodiment of the sensor pad assembly 15. The LVDS ICs 301 allow for differential signals to run between the control box and the sensor pad and for increased distance between sensor pad assemblies 15 and a control box 50. A flexible PCB 304 provides support for electrical traces 303 and accelerometers 5, without interfering with flexibility and mobility. A rigid PCB 302 provides support for the LVDS ICs 301, the CPLD (complex programmable logic device) 306 and voltage regulators 307. The CPLD (or other logic such as a SerDes (serializer/deserializer)) 306 provides logic to the accelerometers and collects data to be sent to the control box. The voltage regulators 307 ensure that the voltage levels are appropriate for the electronics and sensors in the sensor pad assembly. The cable to control box connector 308 is a flexible multi-conductor cable between the sensor pad assembly and control box.

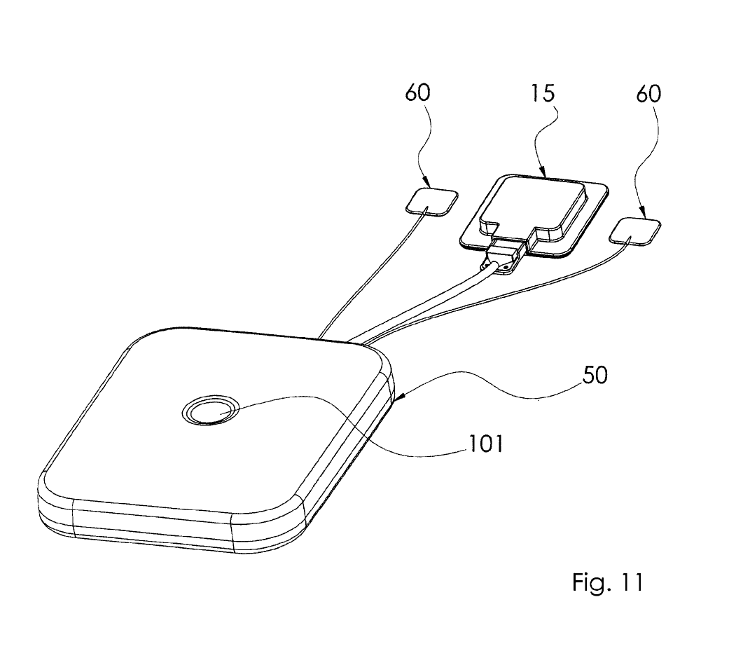

FIG. 11 shows a sensor pad assembly 15 of the third embodiment of the present invention and a pair of electro-stimulation electrodes 60 connected to a control box 50.

In an embodiment of the system of the invention depicted in FIG. 7, a sensor pad assembly 15 and two electro-stimulation electrodes 60 are placed on an axial or appendicular muscle, and a sensor pad assembly 15 and two electro-stimulation electrodes 60 are placed on the lateral pair of that axial or appendicular muscle. In the example shown in FIG. 7, the muscles are leg muscles. Each sensor pad assembly 15 and each electrode 60 are connected to a control box 50 (connections not shown). Software or an app in a local device 20, 30 or 40 controls the control box 50, specifically the electro-stimulation electrodes 11 and the sensor pad assembly 15 data acquisition and collection. A hosted cloud application in the cloud 10 stores and computes data acquisition for report generation, although local computing devices can also be used for this function.

The device and system of the present invention measures muscle contractile properties. Changes in muscle function, specifically muscle activation patterns, muscle abnormalities, muscle symmetry, muscle synchronization, muscle tone, muscle force and muscle fatigue are measured. The quantitative data obtained from the invention provides assessments and monitoring of individual's muscle function and changes that result from various interventions, to enable diagnosis, assessment, and treatment.

The sensor pad assembly 15 and system of the present invention measures and assesses superficial skeletal muscle contraction properties, specifically involuntary muscle twitch mechanical response properties which may be used to determine muscle function abnormalities, muscle activation patterns, muscle symmetry of lateral muscle pairs, muscle synchronization of antagonist muscle, muscle force, muscle acceleration, muscle velocity, muscle tone, muscle fatigue, muscle power/torque and muscle efficiency.

The use of the sensor pad assembly of the present invention:
a. Automates detection of the area with the greatest mechanical contraction within a superficial skeletal muscle;
b. Measures mechanical contractile properties in involuntary stimulated superficial skeletal muscles; and
c. Measure states of submaximal, maximal, and supramaximal muscle contractions.

Each sensor pad assembly 15 allows for quick placement on a superficial skeletal muscle selected to be assessed. The use of paired sensory pad assemblies in the superficial skeletal muscle response measuring system of the present invention allows information to be gathered simultaneously on lateral muscle pairs for comparative analysis and faster assessment. Involuntary contraction of the muscle being assessed is achieved with electrical stimulation using an automated ramping protocol (described in more detail below). The contracting muscles produce a distinct twitch response that is formatted into a muscle response graph consisting of a latent phase, contraction phase and relaxation phase. Data from the various phases of the twitch response are analyzed using algorithms that generate metrics that can later be displayed in report format. The metrics include measurements consisting of forces, acceleration, velocities, and distance.

These metrics are used to calculate other properties of muscle contraction that include power, work, torque, momentum, tone/tension, efficiency, fatigability, fiber type composition. Different types of contraction intensities are produced to assess submaximal, maximal, and supramaximal states of function. The metric values are compared (1) within the subjects own database of lateral muscle pair and agonist-antagonist muscle pairs, and (2) compared against population-specific reference databases of lateral muscle pairs and agonist-antagonist muscle pairs. Contextual information pertaining to the individual person, sorts the subject into populations for accurate comparisons to be made as well as providing additional insight into other physiological factors that could affect muscle function between tests.

The control box 50 has two channels of stimulation and two channels of measurement to be able to assess two muscles simultaneously. In an embodiment of the present invention, the control box 50 connects wirelessly to any smart device 40 with Bluetooth®, or in the alternative, to connect/activate via WiFi. The smart device 40 operates a downloadable app to connect to and manipulate the control box 50. The smart device 40 communicates with a hosted cloud application that applies the algorithms that calculate the metrics, store the data, and generates reports which are sent back to the smart device 40 for viewing. In an embodiment of the invention, the hosted cloud application has e-portal accessible through a browser that allows manipulation of the data, generation of additional reports, create/edit subject information, and different formats of data import/export features.

The sensor pad assembly 15 of the present invention measures response to electro-stimulation of a muscle, with data measured and collected. The automation aspect of the present invention includes (1) a sensor pad that is large enough to be placed by an individual with minimal knowledge of muscle anatomy upon the muscle of interest; (2) an algorithm that detects the area of greatest mechanical movement occurring in a vector summation of x, y, and z axis that determines the usable data to transfer to local app and cloud; (3) an automated ramping protocol that brings the muscle to various states of submaximal, maximal, and supramaximal contractions; (4) wireless communication between control box 50 and smart device 40; (5) wireless communication between smart device 40 and cloud application 10; (6) processing of raw data on cloud by algorithms; (7) wireless communication between cloud application and smart device 40; (8) display of processed and computed data in report format.

The sensor pad design allows placement over top of superficial skeletal muscle of primary interest to provide coverage of the muscle belly with minimal anatomical knowledge required. Placement may be assisted using anatomical pictures of placement provided on the local smart device application during administration of the test.

The sensor pad multi-accelerometer array automates the process of determining the specific area of the muscle belly that provides the most optimal muscle response measurement by collecting and analyzing data from the accelerometer sensors and choosing only the data from the accelerometers sensing the greatest mechanical contraction. The process of identifying the area of greatest mechanical contraction uses an algorithm that processes the raw data on the control box firmware to identify which of the accelerometers exist within a certain diameter of the muscle belly exhibiting the largest resultant vector of measurement in a three dimensional plane of movement (x, y, z) following a single pulse of stimulation. The average of the resultant vector of X number of chosen accelerometers then provides a primary muscle response graph upon which additional metrics are calculated.

For example, in an embodiment of the invention there are sixteen accelerometers spaced twelve mm center to center or nine accelerometers spaced 17 mm by 17 mm center to center in a 50 mm by 50 mm sensor pad, which provides a high likelihood that three accelerometers will be over the representative area providing most useful muscle data, and at least one accelerometer will be over the representative area to provide a maximal and accurate readings. As such, this 50 mm by 50 mm sensor pad with sufficient accelerometers provides reliable results whereas a lesser number of accelerometers on the sensor pads can work in this superficial skeletal muscle response measuring system of the present invention but have less reliability or require an operator with more training. More accelerometers on the sensor pads are acceptable in the superficial skeletal muscle response measuring system of the present invention, but too many may become costly and unwieldy.

In an embodiment of the present invention, using a sensor pad with nine or sixteen accelerometers, an algorithm is used to identify accelerometers that fit the inclusion criteria of maximal force, acceleration, velocity and distance. A vectoring algorithm combines each of the accelerometer x, y, z axis results. In the cloud (or alternatively locally) software an averaging algorithm averages the accelerometer's x, y, z data that fits the inclusion category to combine into one single measurement of force, acceleration, velocity and distance which gives a single measurement of left side and right side in force, acceleration, velocity and distance. After repeated tests this generates four muscle response graphs: (I) force graph, (II) acceleration graph, (III) velocity graph, (IV) distance graph for every accelerometer selected for data computation. Each axis is calculated into a resultant vector for each of these muscle response graphs. A final muscle response average of all selected accelerometers for every graph is then used to generate final metrics.

The control box of the superficial muscle response measuring system of the present invention with nine accelerometers comprises software with a first algorithm to identify from said nine accelerometers of each of said sensor pads a selected group ranging from one to nine accelerometers from each of said sensor pads with the most reliable force, acceleration, velocity and distance data, and a second algorithm combining data from the x, y, z axis of each of said selected group of from each of said sensor pads to produce three combined results of each of force, acceleration, velocity and distance. As such there is a single measurement of force, acceleration, velocity and distance for the left side sensor pad and a single measurement of force, acceleration, velocity and distance for the right side sensor pad,

Further, there is computer software hosted on the cloud with an algorithm for characterizing and averaging the data from at least two of three combined results into a single result of force, acceleration, velocity and distance for the left sensor pad and a single result of force, acceleration, velocity and distance for the right sensor pad.

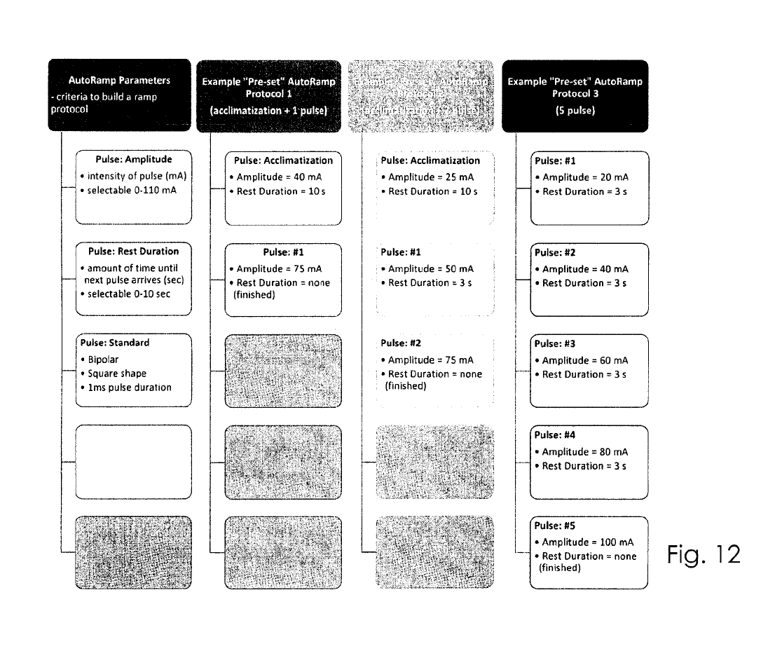

The automated ramping protocol is a custom selection of single pulse criteria to bring the muscle to a certain desirable level of muscle contraction. This single pulse is initially as comfortable as possible for the person being treated. Each additional pulse of stimulation further brings the muscle closer to the desired state of assessment (submaximal, maximal, supramaximal) with pre-established cut-off criteria. There is a custom selection of (a) starting pulse amplitude, (b) rate of pulse amplitude increase at each interval, (c) finishing pulse amplitude (cut-off criteria), (d) pulse duration, (e) rest interval (between pulses), and (f) total # of pulses. Data produced is used either as a rate of change of each muscle response (comparison between metrics), or calculated at the final muscle response when the desired threshold has been achieved (submaximal, maximal, supramaximal). An example of an automated electro-stimulation ramping protocol is provided in FIG. 12.

Wireless communication between the control box 50 and the smart device 40 is via any means available, for example, a Bluetooth® connectivity protocol that transmits and set-ups WiFi protocols. In the embodiment shown in FIG. 9, there is control of the control box 50 via Bluetooth®, and alternatively via WiFi. Control and interface of the control box is via a local downloadable app or software depending on the device being used. For example, wireless communication between a smart device 50 and a hosted cloud application 10 is via the smart device's connectivity with the internet (WiFi or service provider).

Upon the completion of the test (X # of muscles), the user selects from the smart device local application a desired format for report generation. There are selectable, pre-generated and custom options for data presentation in report format and other options to export the data in various formats for incorporation into other software. After algorithm calculation has been completed, the data comes back from the hosted cloud app in the requested format and is displayed on the smart device for further interpretation (by the tester).

The final muscle metrics generated can identify muscle function abnormalities, muscle activation patterns, muscle symmetry of lateral muscle pairs, muscle synchronization of agonist-antagonist muscle, muscle force, muscle acceleration, muscle speed, muscle tone, muscle fatigue, muscle power/torque and muscle efficiency.

An individual person using the invention over time can generate a variety of muscle function reports and this allows the individual to monitor trends and changes in muscle function response to various interventions and protocols, e.g. surgery recovery or specific exercise regimens.

In an embodiment of the invention, data is collected from nine or sixteen accelrometers from two sensor pad assemblies which generate data from eighteen or thirty-two accelerometers, respectively, for analysis. This provides a lot of data quickly. Even after the first test, the individual is starting to create a context based reference database.

Table I shows the types of measurements and metrics produced by the device and software.

TABLE I
measurements and metrics produced by the device and software

| Metric Name | Value | Variable | Explanation |
|---|---|---|---|
| Max Muscle Power | “Maximal Force +ve” | (+)Fm | maximal +ve force |
| Max Muscle Breaking | “Maximal Force −ve” | (−)Fm | maximal −ve force |
| Muscle Force | “Force” (undescribed) | F | force measured at different |
| | | | temporal intervals |
| Max Acceleration | “Maximal Acceleration” | (+)Am | maximal +ve acceleration |
| Max Deceleration | “Maximal Deceleration” | (−)Am | maximal −ve acceleration |
| Muscle Acceleration | “Acceleration” | A | acceleration measured at |
| | (undescribed) | | different temporal intervals |
| Max Velocity/Speed | “Maximal Velocity” | Vm | maximal velocity recorded in |
| Muscle Velocity | “velocity” (undescribed) | V | velocity measured at different |
| | | | temporal intervals |
| Max | “Maximal Displacement” | Dm | maximal displacement/distance |
| Displacement/Distance | | | |
| Displacement/Distance | “Displacement/Distance” | D | displacement/distance measured |
| | | | at different temporal intervals |
| Muscle Power | “Acceleration | (+)At(T) | total time spent in acceleration |
| Generating Period | Interval” | | |
| Muscle Breaking Period | “Deceleration | (−)At(T) | total time spent in deceleration |
| | Interval” | | |
| Acceleration Period | “Time to Maximal | At | interval of time spent during a period of |
| | (+) A” | | acceleration generation |
| Deceleration Period | “Time to Maximal | | interval of time spent during a period of |
| | (−) A” | | deceleration generation |
| Velocity/Speed Period | “Velocity Interval” | Vt(T) | total time spend in velocity |
| Velocity/Speed Period | “Time to Maximal | Vt | interval of time spend during a period of |
| | V” | | velocity generation |
| Displacement/Distance | “Contraction Time” | Dt(T) | total time spent in displacement |
| Period | | | |
| Displacement/Distance | “Displacement | Dt | interval of time spent during a period of |
| | Interval” | | displacement generation |

a) Force measurements; in units of g's (earth's gravity). Software is used to identify maximal forces that occur during certain time intervals during recorded contraction.
b) Acceleration measurements; in units of m/s/s. Software is used to identify maximal accelerations and decelerations that occur during certain time intervals during recorded contraction.
c) Velocity measurements; in units of m/s. Software is used to identify maximal velocities that occur during certain time intervals during recorded contraction.
d) Distance measurements; in units of mm. Software is used to identify maximal distances and displacements that occur during certain time intervals during recorded contraction.
e) Time measurements; in units of ms. Software is used to identify durations of time spent in various indicated states of measurement during recorded contraction.

Table II is a table showing comparisons that can be made with measurements and metrics of the present invention.

TABLE II
comparisons of measurements and metrics from Table I

| Comparisons | Concept | Explanation |
|---|---|---|
| Muscle Pairs | Lateral symmetry | L to R comparison of muscle metrics |
| Joint Pairs | Joint synchronization | agonist to antagonist comparison |
| Kinetic Chains | Functional synchronization | agonist-synergist-stabilizer comparison |
| Relative - Individual | Comparison between tests | within subject, tracking changes |
| Relative - Group | Comparison between subjects | between subjects, tracking differences |
| Reference | Comparison against population | between populations with selectable |
| | | characteristics |
| Norms | Definition of “healthy” | identification of parameters that define |
| | | a healthy population |
| Diagnostic | Definition of “unhealthy” | identification of parameters that |
| | | deviates from normal that result in a true |
| | | positive gold standard test against the |
| | | diagnostic criteria |
| Abnormal | Definition of “dysfunctional” | identification of parameters that |
| | | deviate from normal but result in a |
| | | negative gold standard test against the |
| | | diagnostic criteria |

a) Lateral symmetry of muscle pairs
b) Joint synchrony of co-contracting agonist-antagonist muscles
c) Functional synchrony of kinetic chain muscles
d) Relative within subject between assessments taken at different times/dates
e) Relative between subjects that share similar characteristics (selectable)
f) Against reference populations that share similar characteristics (selectable)
g) Against reference populations of normal that are deemed healthy
h) Against reference populations of abnormal that are deemed unhealthy (in absence of traditional medical tests and imaging)
i) Against reference populations of abnormal that are deemed injured or diseased (in present of traditional medical tests and imaging)

The measurement results provide data of skeletal muscle contraction in submaximal, maximal, and supramaximal involuntary states, which allows comparison within an individual of relative lateral muscle pair and agonist-antagonist mechanical contractile properties (see table II), and with data of multiple individuals on a cloud-based reference database of occupation, lifestyle and sport specific populations of relative lateral muscle pair and agonist-antagonist mechanical contractile properties etc. allows comparison with other individuals and benchmarking.

While embodiments of the invention have been described in the detailed description, the scope of the claims should not be limited by the preferred embodiments set forth in the examples, but should be given the broadest interpretation consistent with the description as a whole.

What is claimed is:

1. A superficial skeletal muscle response measuring system comprising: two sensor pads each with a plurality of three-axis accelerometers capable of measuring muscle contraction responses to stimulation; a pair of electro-stimulation electrodes for each sensor pad; a control box having capability to send stimulation to said electro-stimulation electrodes and to process and transmit data when muscle contraction responses are received from said sensor pads.

2. The superficial muscle response measuring system of claim 1 in which the sensor pads are each 50 millimeters by 50 millimeters.

3. The superficial muscle response measuring system of claim 2 in which the sensor pads each have nine three-axis accelerometers arranged in a three by three array.

4. The superficial muscle response measuring system of claim 2 in which the sensor pads each have sixteen three-axis accelerometers arranged in a four by four array.

5. The superficial muscle response measuring system of claim 1 in which the three-axis accelerometers are capable of measuring submaximal, maximal and supramaximal muscle contraction responses to stimulation.

6. A sensor pad comprising a circuit board with a three by three array of nine accelerometers, a housing body in which the circuit board rests, and a protective covering over the circuit board.

7. The sensor pad of claim 6, in which the protective covering additionally comprises nine cells within which said accelerometers fit.

8. The sensor pad of claim 7, in which the accelerometers are spaced seventeen millimeters apart center to center, and the sensor pad additionally comprises an adhesive layer under the housing body, said adhesive comprising the quality of adhering to and releasing from skin.

9. A sensor pad of claim 6, wherein the circuit board is carved out to further comprise slots around each accelerometer wherein each accelerometer rests on a cantilever arm.

10. The sensor pad of claim 9, in which the protective covering additionally comprises nine cells within which said accelerometers fit.

11. The sensor pad of claim 10, in which the accelerometers are spaced seventeen millimeters apart center to center, and the sensor pad additionally comprises an adhesive layer under the housing body, said adhesive comprising the quality of adhering to and releasing from skin.

12. A sensor pad of claim 11, which further comprises corresponding slots in the covering, the housing body, and the adhesive layer which are contiguous with the slots in the circuit board.

13. A sensor pad comprising a circuit board with a four by four array of sixteen accelerometers, a housing body in which the circuit board rests, said housing body having sixteen cells in which said accelerometers fit within when the circuit board is resting in the housing body, and a protective covering over the circuit board.

14. The sensor pad of claim 13, in which the accelerometers are spaced twelve millimeters apart center to center, and the sensor pad additionally comprises a cap with an adhesive border which cap fits over and around said circuit board resting in said housing body, and said adhesive border is capable of removably adhering to skin.

15. A control box comprising a first connector for connecting to a first pair of electrodes, a second connector for connecting to a second pair of electrodes, a third connector for connecting to a first sensor pad with multiple accelerometers capable of measuring superficial muscle contraction responses, and a fourth connector for connecting to a second sensor pad with multiple accelerometers capable of measuring superficial muscle contraction responses, wherein said control box is capable of controlling the delivery of stimulation to said two pairs of electrodes and receiving muscle contraction responses from said two sensor pads.

16. The control box of claim 15, wherein the accelerometers are 3-axis accelerometers and the sensor pads are capable of measuring at intervals within contraction and relaxation phases of a muscle contraction response.

17. The superficial muscle response measuring system of claim 1, in which said control box further comprises software with a first algorithm to identify from said accelerometers of each of said sensor pads selected accelerometers from each of said sensor pads with the most reliable force, acceleration, velocity and distance data, and a second algorithm combining data from the x, y, z axis of each of said selected accelerometers from each of said sensor pads to produce three combined results of force, acceleration, velocity and distance.

18. The superficial muscle response measuring system of claim 17, further comprising computer software hosted on the cloud with an algorithm for characterizing and averaging the data from at least two of three combined results into a single result of force, acceleration, velocity and distance for each of said sensor pads.

19. Use of the superficial muscle response measuring system of claim 18, to determine muscle function normalities and abnormalities, muscle activation patterns, lateral symmetry of muscle pairs, muscle co-contraction of agonist-antagonist muscles, muscle force, muscle acceleration, muscle velocity, muscle distance, muscle tone, muscle fatigue, and muscle efficiency.

20. A sensor array comprising a circuit board with an array of accelerometers, wherein the circuit board is carved out to further comprise slots around each accelerometer wherein each accelerometer rests on a cantilever arm.
